(19) United States
(12) Patent Application Publication
(10) Pub. No.: US 2008/0276945 A1
(43) Pub. Date: Nov. 13, 2008

(54) Lateral sliding adjustable earplug

(76) Inventor: Leon D. Rosen, Santa Monica, CA (US)

Correspondence Address: LEON D. ROSEN;FREILICH, HORNBAKER & ROSEN, Suite 1220, 10960 Wilshire Boulevard, Los Angeles, CA, 90024, US

(21) Appl. No.: 11/801,969

(22) Filed: May 11, 2007

(51) Int. Cl. A61F 11/00

(52) U.S. Cl. 128/864

(57) ABSTRACT

An earplug has an earplug body (12) with a front sealing portion (20) that seals to a person's ear canal, a body rear portion (22) that lies out of the ear canal, and a passage (26) that extends along the body axis and that allows environmental sound to flow into the ear canal when the passage is not blocked. The body rear portion has a lateral slot (30), and the earplug includes a blocking element (14) that is laterally (L) slidable along the slot. The blocking element can slide between a first position (14A) wherein a hole (32) in the element is not aligned with the passage to prevent sound from reaching the ear canal, and a second position (14B) wherein the hole is aligned with the passage to allow sound to reach the passage.

### BACKGROUND OF THE INVENTION

Workers who work in noisy environments usually wear earplugs that block almost all sound, to protect their hearing. Occasionally, someone tries to speak to the worker, and this has usually required the worker to remove an earplug and thereafter reinstall it in his/her ear canal. The reinstallation of earplugs in a workman's ear canal has disadvantages, because it often occurs away from a clean environment. One type of earplug is of slow recovery foam, and it requires the worker to roll the earplug between his/her fingers to a small diameter to insert it. If the worker has dirty fingers then the earplug may spread dirt into the ear canal. Other types of earplugs are formed of ordinary foam with a stiffener, or are formed of soft rubber, but removing and replacing them in the ear canal can dirty the earplugs and ear canal, and is often time consuming.

One type of earplug that allows a worker to temporarily hear and then block noise without taking the earplug out of his/her ear, is described in U.S. Pat. No. 5,332,871. In that patent, the earplug body has an axial passage, and a valve guide is attached to the rear end of the earplug body to block or unblock the passage. A controlling part is slidable on the valve guide between a position aligned with the valve guide passageway so the controlling part blocks sound, and a position out of alignment with the passageway to allow sound to pass. However, that earplug has several disadvantages.

One disadvantage of the earplug of the above-referenced patent is that the valve is complicated as it requires fitting the rear end of an earplug body into a large hole in the valve guide and fixing it thereat, and it uses a separate controlling part that is slidably mounted on the valve guide, all of which increases the cost of the earplug. A worker may have trouble determining the exact position to which the earplug should be slid for maximum noise blocking. The valve is wide, and may not fit comfortably into a person's outer ear. An earplug that could be easily changed from blocking to unblocking, that was of very simple design, that enabled easy use, and that was of small size, would be of value.

### SUMMARY OF THE INVENTION

In accordance with one embodiment of the invention, an earplug is provided that can switch from blocking to unblocking sound while still lying in a person's ear canal, which is of simple and low cost construction, which is easily switched, and which is compact. The earplug includes an earplug body with a front sealing portion that seals to a person's ear canal, and a rear body portion that lies outside the ear canal. The rear body portion has a laterally-extending slot, and a blocking element can slide in the slot. The blocking element has a hole, and the blocking element can slide between a first position where its hole is in line with the earplug body passage to pass sound, and a second position wherein its hole is completely out of line with the earplug body passage to block sound. By constructing the blocking element with a hole that slides into and out of alignment with the body passage as the blocking element slides, instead of constructing the blocking element with a small blocking part that slides over or away from the body passage, applicant can use a simple blocking element that slides in a slot in the earplug body rear portion

The hole in the blocking element can be elongated with one end tapered in width along the length of the blocking element, so a person can adjust the level of sound blocking. A controlled amount of sound blocking allows a person to block only enough sound for comfort, as when listening to a loud rock-and-roll concert. The opposite ends of the blocking element that slide in a slot of the body rear portion, can be constructed so only a first end of the blocking element projects from a side of the earplug body when there is complete blocking, and only the opposite second end of the blocking element projects from the opposite side of the earplug body when there is complete unblocking. The opposite ends of the blocking element have different feels, as where one is flat and the other is semi-pointed.

The novel features of the invention are set forth with particularity in the appended claims. The invention will be best understood from the following description when read in conjunction with the accompanying drawings.

### BRIEF DESCRIPTION OF THE DRAWINGS

FIG. 1 is a rear isometric view of an earplug of the present invention.

FIG. 2 is a sectional side view of the earplug of FIG. 1 with the blocking element in a blocking position.

FIG. 3 is a view similar to that of FIG. 2, but with the blocking element in an unblocking position, and showing the earplug installed in a person's ear canal.

FIG. 4 is a rear view of the blocking element and a phantom view of the earplug, with the blocking element in a blocking position.

FIG. 5 is a view similar to that of FIG. 4, but with the blocking element in a unblocking position.

FIG. 6 is a view similar to that of FIG. 4, but with the hole in the blocking element being elongated and having one end tapered, and lying in a blocking position.

FIG. 7 is a view similar to FIG. 6 but with the blocking element and its hole lying in a partial blocking position.

FIG. 8 is a sectional side view of an earplug of another embodiment of the invention.

FIG. 9 is a sectional view of an earplug similar to that of FIG. 2, but with a sound filter in the passage with the view taken perpendicular to that of FIG. 2.

### DETAILED DESCRIPTION OF THE INVENTION

FIG. 1 illustrates an earplug 10 of the invention, which includes an earplug body 12 and a blocking element 14. The earplug body has a front sealing portion 20 that can be pushed forward F into a person's ear canal to block it, and includes a rear portion 22 that lies rearward R of, and therefore outside of, the person's ear canal. The earplug body has an axis 24 and has a passage 26 extending along the axis. The rear portion of the earplug body has a slot 30 extending laterally L therein. The blocking element 14 is slidable in lateral directions L within the slot, and has a through hole 32.

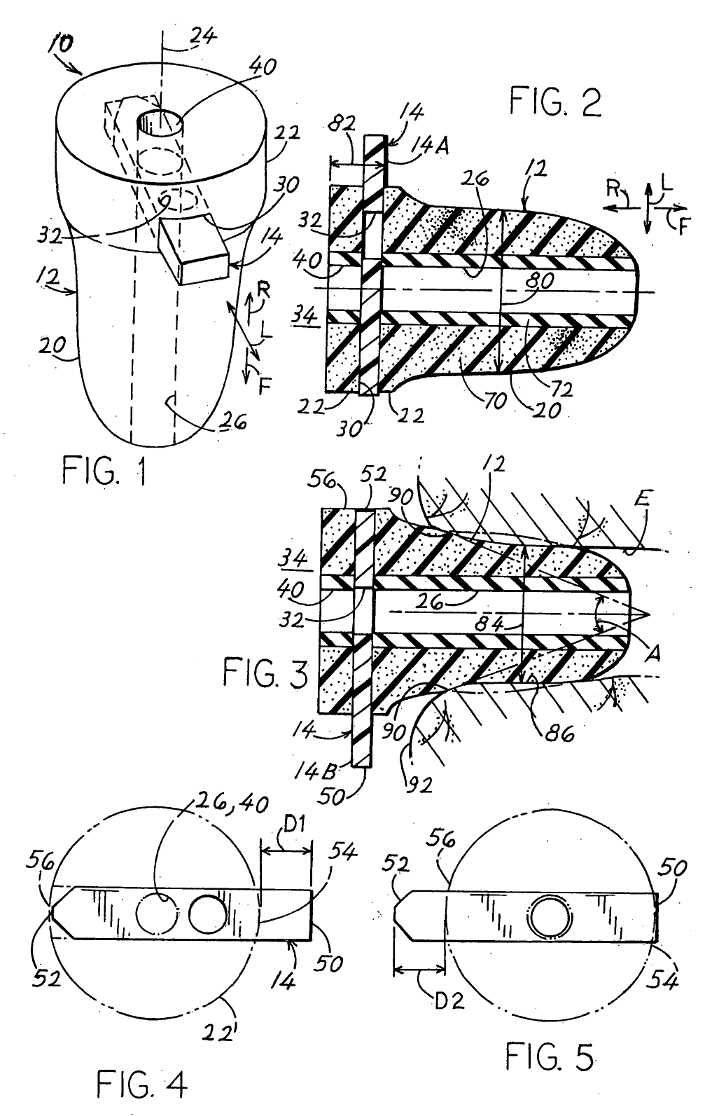

FIG. 2 shows the blocking element 14 in a first position 14A at which the element hole 32 is completely or fully out of line, or alignment with the earplug body passage 26 so no part of the hole is aligned with the passage. Sound in the environment 34 can pass though a rear part 40 of the passage that lies rearward of the slot 30, but most of such sound is stopped by the blocking element 14. FIG. 3 shows the earplug body 12 in a person's ear canal E, but with the blocking element in a second position 14B at which the element hole 32 is aligned with the earplug passage 26, so a high portion of sound in the environment 34 that has passed though the passage rear part 40, passes, or flows into the person's ear canal E.

The blocking element 14 shown in FIGS. 4 and 5 has opposite ends 50, 52. In the element first position, shown in FIGS. 2 and 4, the first end 50 projects a distance D1 from a first lateral side 54 of the earplug body, but the second end 52 does not project, or does not project as far, out of the second side 56 of the earplug body. In the element second position, shown in FIGS. 3 and 5, the second end 52 of the element projects a distance D2 from the second side of the earplug body, but the first end 50 of the element does not project or project as far out of the first side 54 of the earplug body. A person wearing the earplug, with the blocking element in the first, or blocking position of FIGS. 2 and 4, can easily switch to the unblocking position of FIGS. 3 and 5 by pressing in the first end 50 of the blocking element until the first end 50 is about flush with the first side 54 of the earplug body. Then, the person can switch back to the blocking position by pressing in the second end 52 of the blocking element until it is flush with the second side 56 of the earplug body.

The opposite ends of the blocking element 14 preferably have different shapes that feel different, to make it easy for a person to determine that one end is the end to be pushed in to block or unblock the earplug passage. Actually, the person will usually press in the end that projects a lot from the earplug body to switch between blocking and unblocking, although the ability to distinguish one end from the other by feel can be useful.

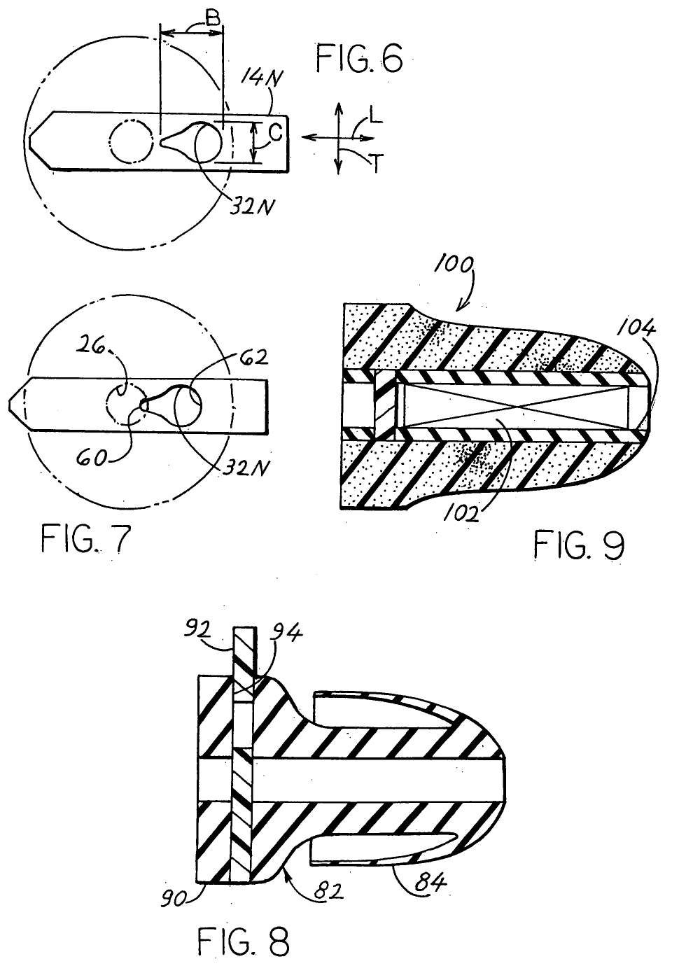

In some situations, a person wants to indefinitely block most sound in the environment, but still let in a considerable amount, or considerable percent, of sound which is much more than would flow in if the earplug were in its first, or blocking, position. FIG. 6 shows an earplug with a hole 32N in the blocking element 14N, that is elongated in a lateral direction L that is parallel to its direction of sliding, and that is of tapered transverse T width. That is, the lateral length B of the hole is greater than its transverse width C. In FIG. 7, only a second end portion 60 of the hole 32N is aligned with the earplug body passage 26, so a small portion of environmental sound flows to the ear canal. In FIG. 7, the percent of environmental sound that reaches the ear canal is greater than in the blocking position of FIG. 6 but is smaller than in an unblocking position wherein a first end portion 62 of the hole lies aligned with the passage. The ability to hear a moderate amount of environmental sound can be useful, for example, for a person attending a loud rock-and-roll concert, who wants to clearly hear the music but not hurt his/her hearing. Another example is where a plant foreman works in an only moderately noisy environment, where blocking only a moderate portion of sound will protect his hearing and allow him to hear machinery and people who are talking loudly. It should be noted that the hole 32, 32N in the blocking element can be formed as a cutaway in one side edge of the element instead of the hole being completely surrounded by material of the element.

FIGS. 1-6 show an earplug body 12 (FIG. 2) that includes an outer body portion 70 of soft foam and a stiffener 72 of a much stiffer but elastic material such as rubber. There are many other forms of earplug bodies with sealing portions that seal to a person's ear canal. FIG. 8 shows an earplug 80 with an earplug body 82 formed of solid elastomeric material (an elastic, or elastomeric material is one with a Young's modulus of elasticity of no more than 50,000 psi). The earplug has at least one flange 82 that is thin and long to seal to a person's ear canal. The earplug body has a rear portion 90 and has a blocking element 92 that slides in a slot 94 of the body rear portion.

There are a wide range of materials that can be used for the earplug body and for the blocking element. It is desirable that the blocking element such as 14 in FIG. 1 form a tight seal with the earplug body to provide very good noise blocking in the blocking position of the element. This usually requires a close fit (preferably an interference fit) of the sealing element with the walls of the slot 30 in the earplug body. In FIG. 2, the blocking element 14 is sealed to the solid elastomeric material of the earplug body stiffener 72, so the blocking element can be formed of rigid engineering plastic or of elastomeric material, so long as a tight fit is achieved that still allows the blocking element to slide. The tight fit is also desirable to hold the element in whatever position it has been slid to. Since the blocking element projects only a short distance beyond either side of the earplug body, the element can be formed of an elastomeric material and still be slid back and forth.

As mentioned above, the blocking element 14 of FIG. 1 projects only a small distance beyond either side of the earplug body in its blocking and unblocking positions. The lateral projection distance such as D1 and D2 in FIGS. 4 and 5, is usually no more than half the diameter of the rear portion 22 of the earplug body. This is desirable because it allows the rear end of the earplug to lie in the wearer's outer ear with minimum or no interference with walls of the outer ear. Each element end preferably projects by no more than one-third the body rear portion diameter when the opposite element end is flush with the body rear portion. The ear canals E of most people range between about 0.27 inch and 0.33 inch, with the average being about 0.30 inch (7.6 mm). The earplug outer body sealing portion 70 (FIG. 2) has an initial diameter at 80 such as 0.4 inch and is compressed to about 0.3 inch when installed. The earplug rear portion 22 has a diameter of about 0.6 inch, and extends rearwardly a distance 82 of about 0.2 inch further than an earplug of this type (e.g. as described in U.S. Pat. No. 4,774,938) without sound adjustment. The blocking element 14 has a lateral length of about 0.75 inch. The maximum lateral width (about 0.75 inch) of the earplug at the blocking element, is no more than 250% the diameter 84 (FIG. 3) of a person's ear canal, and the width (0.60 inch) in a perpendicular direction is only 200% of the diameter of the ear canal. The width of a person's ear canal can be considered to be the width of a sealed ear canal location 86 (FIG. 3, which has a diameter of about 0.3 inch as mentioned above) located about 0.3 inch forward of a location 90 where opposite sides of the ear canal diverge at an angle A of 45°. The relatively small width of the earplug rear portion 22 with the blocking element therein, allows them to better fit in the outer ear part 92 that lies immediately outside the ear canal.

In most cases, the earplug is used by a worker in a noisy environment and is unblocked by the worker primarily to hear a person who is shouting at him. FIG. 9 shows another earplug 100 that is similar to the earplug of FIGS. 1 and 2, but that has a sound filter 102 lying in and blocking its passage 104. The sound filter allows the passage of sound of a limited frequency band that includes voice and that enables voice to be understood, when the blocking element is in its unblocking position. However, even in the unblocking position, other frequencies are blocked.

The useable voice frequency band ranges from about 300 Hz to 3400 Hz. However, if narrower band of frequencies (e.g. 300 Hz to about 900 Hz) is passed to the ear canal, then a voice can be understood even though it sounds filtered. Applicant prefers to limit the pass band to less than about 1700 Hz, so even when the earplug is unblocked most high frequency sound (e.g. above about 2000 Hz or 4000 Hz) is still blocked (that is, the volume is attenuated by more than 3 db below the voice band).

Thus, the invention provides an ear plug that enables the unblocking and blocking of a person's ear canal without requiring the earplug to be removed from the ear, that is of simple and low cost design, that is easily operated to change configuration, and which has a compact rear portion for more easily fitting into a person's ear canal. The earplug includes an earplug body with a passage extending primarily along its axis, and a blocking element that is slidable in a lateral direction between blocking and unblocking positions. The blocking element has a hole that is moved out of alignment with the earplug passage to block it, rather than the blocking element being a solid part that is moved into alignment with the passage to block it. The hole in the blocking element can be formed as an elongated hole with a tapered end, to more easily enable fine adjustment of the degree of sound blockage.

Although particular embodiments of the invention have been described and illustrated herein, it is recognized that modifications and variations may readily occur to those skilled in the art, and consequently, it is intended that the claims be interpreted to cover such modifications and equivalents.

What is claimed is:

1. An earplug with a body that has an axis and a passage extending parallel to its axis, that has a body front sealing portion that fits in and seals to the walls of a human ear canal, and that has a body rear portion that lies outside the ear canal, wherein:
said body rear portion has a slot that extends in a lateral direction which is primarily perpendicular to said axis and that intersects said passage; and including
a blocking member with a hole, said blocking member being moveable along said slot between an unblocking position wherein at least part of said hole is aligned with said passage so said blocking member does not completely block said passage, to thereby allow sound to flow from the environment and through said passage to the ear canal, to a blocking position wherein said hole is completely out of alignment with said passage and said blocking member blocks said passage to prevent almost all sound in the environment from reaching the ear canal.

2. The earplug described in claim 1 wherein:
said body front sealing portion lies in an ear canal and seals to a sealed ear canal location of predetermined diameter, and in one position of said blocking member in said slot, a maximum lateral dimension of said earplug outside the ear canal is less than 250% of said sealed earplug location.

3. The earplug described in claim 1 wherein:
said slot extends along a lateral direction and said blocking member is slidable in said lateral direction along said slot;
said hole in said blocking member is elongated along said lateral direction with said hole having a maximum diameter in a transverse direction that is perpendicular to said lateral direction and to said axis, and said hole has a first lateral end that is tapered in transverse width.

4. The earplug described in claim 1, including:
a filter lying in and blocking said passage, said filter blocking sound above a frequency of 3400 Hz but allowing at least 3 db more of sound of a frequency of 300 Hz to 900 Hz to flow through said passage than sound above 3400 Hz.

5. The earplug described in claim 1 wherein:
in said blocking position a first end of said blocking member projects laterally further out of said body rear portion than an opposite second end of said blocking member projects from said body rear portion, and in said unblocking position said second end of said blocking member projects further.

6. An earplug that has an earplug body with a front sealing portion that fits into a person's ear canal and seals to it, said earplug body having an axis and having a passage extending therealong, and having a mechanism for selectively blocking and unblocking said passage, wherein:
said earplug body is an integrally molded member with a slot therein, and said mechanism comprises a blocking member that is slideably disposed in said slot to slide between first and second positions therein, said blocking member having an opening that is aligned with said passage in said first position to pass sound from the environment through said passage to the ear canal, and that is fully out of alignment with said passage in said second position to block sound.

7. The earplug described in claim 6 wherein:
said blocking member is in the form of an elongated member with opposite ends and with only a first of said ends protruding from one side of said earplug body in said first position and with only an opposite second of said ends protruding from an opposite side of said earplug body in said second position.

8. An earplug with a body that has an axis and a passage extending substantially along its axis, that has a body front sealing portion that fits in and seals to the walls of a human ear canal, and that has a body rear portion that lies outside the ear canal, including:
means in said body rear portion for blocking and unblocking said passage, including an element that has a hole and that is moveable between blocking and unblocking position wherein said hole is respectively aligned and not aligned with said passage, with said element projecting a first distance from a first side of said body rear portion in said first position of said element and projecting less than said first distance from said first side in said second position.

9. The earplug described in claim 8 wherein:
said element projects a first distance D1 from a first side of said body rear portion in said first position of said element, and said element projects less than said first distance from said first side of said body rear portion in said second position.
